(19) United States
(12) Patent Application Publication
(10) Pub. No.: US 2022/0362461 A1
(43) Pub. Date: Nov. 17, 2022

(54) INFUSION DEVICE

(71) Applicants: Inventec Appliances (Pudong) Corporation, Shanghai (CN); Inventec Appliances (Shanghai) Co., Ltd., Shanghai (CN); Inventec Appliances Corp., New Taipei City (TW)

(72) Inventors: Yi-yu LEE, New Taipei City (TW); Wen-tso TSENG, New Taipei City (TW); Hon-wing AU, New Taipei City (TW)

(21) Appl. No.: 17/618,899

(22) PCT Filed: Jun. 14, 2019
(86) PCT No.: PCT/CN2019/091345

§ 371 Date: Dec. 14, 2021

(51) Int. Cl. A61M 5/168; A61M 39/28

(52) U.S. Cl. CPC A61M 5/16813; A61M 39/28; A61M 2205/3317

(57) ABSTRACT

The present invention relates to an infusion device comprising a clamp having a through hole that is configured to allow an infusion tube to pass through therethrough, and further having a baffle wall; a block having a mounting hole and an opening; a pressure member pivoted to the block and configured to press towards the baffle wall and close the infusion tube, wherein a position of the opening corresponds to the pressure member; and a handle pivoted to the block and having a protrusion that is configured to pass through the opening and push up against the pressure member so as to make the pressure member away from the baffle wall when the handle rotates towards the block around an axis, so that the infusion tube is no longer closed by pressing of the pressure member.

### BACKGROUND

### Technical Field

The present invention relates to an infusion device.

### Description of Related Art

In the society moving towards aging and longevity, the demand for medical apparatus is increasing rapidly. Therefore, the quality of medical apparatus has also become a concern. The reliability and safety of medical apparatus is undoubtedly an important topic in the industry.

Generally speaking, it is a common practice to transport medical fluid to patients with infusion device in the medical process. However, during the operation of the infusion pump, every step must be handled with great care to avoid unexpected fluid flowing into the patient's body.

### SUMMARY

One of the objectives of the present invention is to provide an infusion device, which can effectively avoid unexpected flow of fluid medicament in the infusion tube.

According to an embodiment of the present invention, an infusion device comprises:

a clamp having a through hole that is configured to allow an infusion tube to pass through therethrough, and further having a baffle wall;

a block having a mounting hole and an opening, the mounting hole being configured to allow the clamp to at least partially insert and to be connected with the block;

a pressure member pivoted to the block and configured to press towards the baffle wall and close the infusion tube, wherein a position of the opening corresponds to the pressure member; and

a handle pivoted to the block and having a protrusion that is configured to pass through the opening and push up against the pressure member so as to make the pressure member away from the baffle wall when the handle rotates towards the block around an axis, so that the infusion tube is no longer closed by pressing of the pressure member.

In one or more embodiments of the present invention, the infusion device further comprises an elastic assembly which elastically connects the pressure member with the block, wherein the pressure member elastically presses towards the pressure surface before the protrusion pushes up against the pressure member.

In one or more embodiments of the present invention, the elastic assembly is a torsion spring.

In one or more embodiments of the present invention, the handle further has at least a first guide portion, and the block has at least a second guide portion, and when the handle rotates relative to the housing and before the protrusion pushes up against the pressure member, the first guide portion rotates with the handle and catches with the second guide portion to fix relative position of the housing and the block.

In one or more embodiments of the present invention, the first guide portion comprises an arc guide groove, and the second guide portion comprises a guide post that is caught in the arc guide groove when the arc guide groove rotates with the handle.

In one or more embodiments of the present invention, the number of the first guide portions is a pair, which are respectively located at opposite ends of the handle along the axis, and the number of the second guide portions is also a pair.

In one or more embodiments of the present invention, the handle keeps close to the housing when the protrusion pushes up against the pressure member.

In one or more embodiments of the present invention, it further comprises a sensor that is located in the block and a sensed object that is located at the other end of the pressure member configured to press towards the pressure surface, when the pressure member is away from the pressure surface, the sensed object corresponds to the sensor, and the sensor senses the sensed object.

In one or more embodiments of the present invention, the sensor is a Hall sensor and the sensed object is a magnet.

The above embodiments of the present invention have at least the following advantages:

(1) Since the process of allowing the infusion tube to flow the fluid medicament in the block is conducted by the rotation of the handle relative to the housing after the housing is disposed on the block, the unexpected flow of the fluid medicament in the infusion tube can be effectively avoided in the process of the infusion tube being fixing in the block.

(2) The process of allowing the fluid medicament to flow when the infusion device is started after installation of the infusion tube on the block is conducted by a mechanical operation manner that the protrusion of the handle pushes up against the pressure member so as to make the pressure member away from the pressure surface when the handle is pulled. Therefore, the reliability of operation can be effectively improved.

(3) Since the first guide portion of the handle and the second guide portion of the block have been caught with each other so as to ensure that the relative position of the housing and the block has been relatively fixed before the protrusion of the handle pushes up against the pressure member to cause the pressure member away from the pressure surface of the baffle wall, the user, under the condition of reliability and safety, can press the handle to rotate it and make the protrusion of the handle pass through the opening of the block and push it up against the pressure member, so that the pressure member is away from the pressure surface of the baffle wall.

### BRIEF DESCRIPTION OF THE DRAWINGS

In order to more clarify the technical solutions in the embodiments of the present invention, the description of the drawings needed to be used in the embodiments will be briefly introduced below. Obviously, the drawings in the following description are only some embodiments of the present invention. For those skilled in the art, other drawings can also be obtained from these drawings without paying creative work.

FIG. 1 is a partial perspective schematic view illustrating an infusion device according to an embodiment of the present invention.

FIG. 2 is a perspective schematic view illustrating a clamp of FIG. 1 from another viewing angle.

FIG. 3 is a sectional view taken along line A-A of FIG. 1, in which the infusion device comprises a housing and a handle.

FIG. 4 is a sectional view taken along line A-A of FIG. 1, in which the handle rotates and butts against a pressure member.

FIG. 5 is a sectional view taken along line A-A of FIG. 1, in which a protrusion pushes up against the pressure member to keep the pressure member away from a pressure surface.

FIG. 6 is a partial perspective schematic view illustrating the handle and a block of FIG. 1.

FIG. 7 is a partial sectional view taken along line B-B of FIG. 6.

FIG. 8 is a partial sectional view illustrating the infusion device according to another embodiment of the present invention.

### DESCRIPTION FOR THE REFERENCE NUMERALS

100: Infusion device
110: Clamp
111: Baffle wall
112: Pressure surface
113: First catch portion
120: Block
121: Second catch portion
122: Second guide portion
122a: Guide post
130: Pressure member
140: Housing
150: Handle
151: Protrusion
152: First guide portion
152a: Arc guide groove
160: Elastic element
170: Sensed object
180: Sensor
200: Infusion tube
A-A, B-B: Lines
HM: Mounting hole
HO: Opening
HT: Through hole
X: Axis

### DETAILED DESCRIPTION

A plurality of embodiments of the present invention will be disclosed below with the accompanying drawings. For the sake of clarity, many practical details will be described in the following recitation. However, it should be understood that these practical details are not intended to limit the present invention. That is, in some embodiments of the present invention, these practical details are unnecessary. In addition, for the sake of simplifying the drawings, some known and commonly used structures and elements will be illustrated in a simple schematic manner in the drawings. Moreover, if the implementation is possible, the features of different embodiments can be applied interactively.

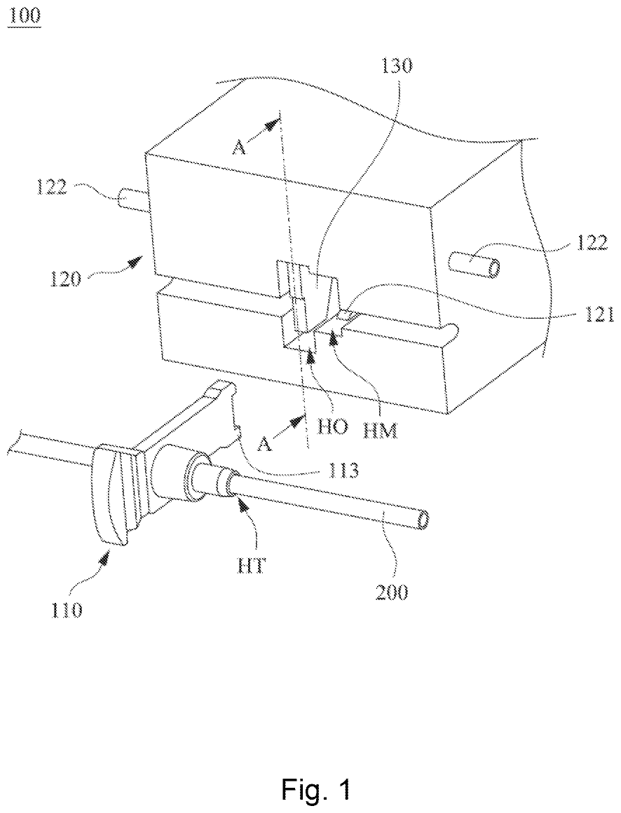

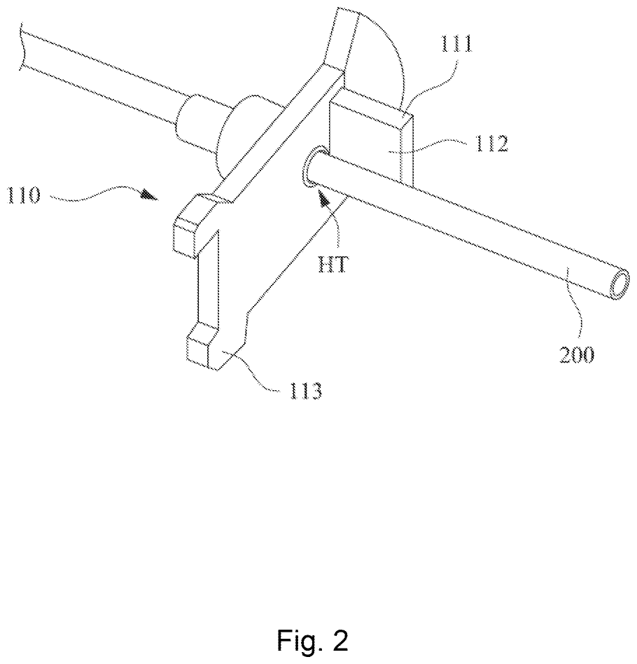

Referring to FIGS. 1-2, FIG. 1 is a partial perspective schematic view illustrating an infusion device 100 according to an embodiment of the present invention, and FIG. 2 is a perspective schematic view illustrating a clamp 110 of FIG. 1 from another viewing angle. In the present embodiment, as shown in FIGS. 1 to 2, the infusion device 100 comprises a clamp 110, a block 120 and a pressure member 130. The clamp 110 has a through hole HT that is configured to allow the infusion tube 200 to pass through, and the infusion tube 200 is adaptable to allow the fluid medicament (not shown) to flow therein. The clamp 110 further has a baffle wall 111 (see FIG. 2) that is adjacent to the through hole HT and at least partially faces the infusion tube 200, and the clamp 110 also has a first catch portion 113. The block 120 has a mounting hole HM that is configured to allow the clamp 110 to at least partially insert, and the block 120 also has a second catch portion 121 that is at least partially located in the mounting hole HM and configured to be fixed with the first catch portion 113. After the clamp 110 is inserted into the mounting hole HM of the block 120, the first catch portion 113 of the clamp 110 and the second catch portion 121 of the block 120 are caught and fixed with each other, so that the clamp 110 can be at least partially fixed in the mounting hole HM of the block 120.

In addition, the pressure member 130 is pivoted to the block 120 and configured to press towards a pressure surface 112 of the baffle wall 111, that is, after the infusion tube 200 passes through the through hole HT of the clamp 110 and the clamp 110 is fixed in the block 120, the infusion tube 200 is at least partially located between the baffle wall 111 and the pressure member 130. Moreover, the infusion tube 200 located between the baffle wall 111 and the pressure member 130 will be flattened being the pressure member 130 pressing towards the baffle wall 111, so that inner walls of the infusion tube 200 keep close to each other, and the fluid medicament cannot flow in the infusion tube 200.

Preferably, a pressure surface 112 is provided on the baffle wall 111, which at least partially faces the infusion tube 200, and the pressure member 130 presses towards the pressure surface 112, so that the infusion tube 200 located between the pressure surface 112 and the pressure member 130 is flattened.

In addition, although in the embodiments of FIGS. 1 and 2, the clamp 110 and the block 120 are fixedly connected through the first catch portion 113 and the second catch portion 121, it should be understood that those skilled in the art can fixedly connect the both by using other similar connection manners and connection structures. The present application should not be limited to the connection manners in the embodiment.

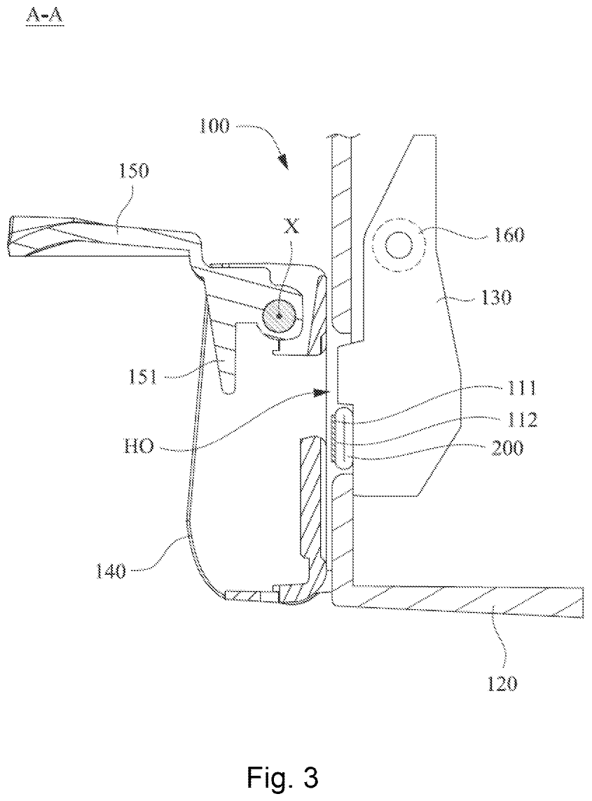

Referring to FIG. 3, it is a sectional view taken along line A-A of FIG. 1, in which the infusion device 100 includes a housing 140 and a handle 150. In the present embodiment, as shown in FIG. 3, the infusion device 100 also comprises a housing 140 and a handle 150. The housing 140 is detachably arranged on the block 120, and the handle 150 is pivoted to the housing 140, and the handle 150 thus can rotate around the axis X relative to the housing 140. Moreover, the handle 150 has a protrusion 151 that at least partially faces the block 120.

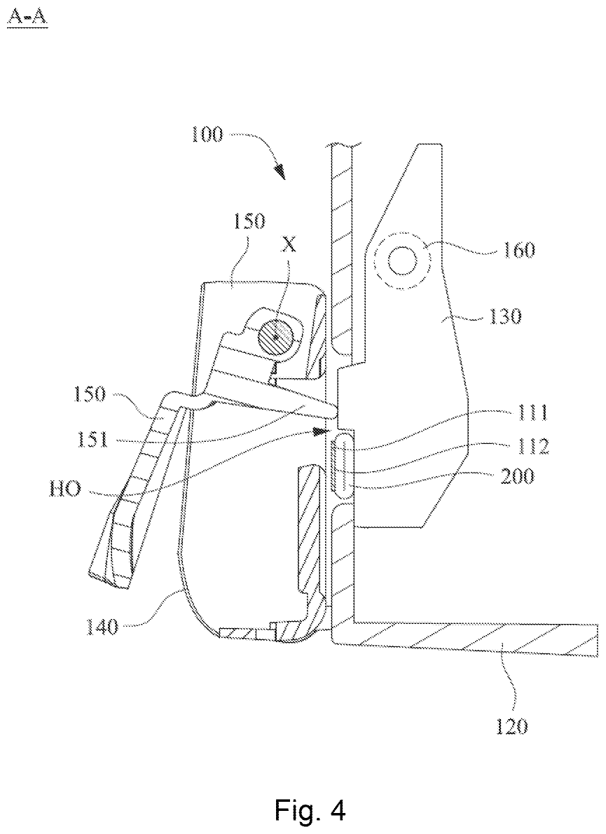

Referring to FIG. 4, it is a cross-sectional view taken along line A-A of FIG. 1, in which the handle 150 rotates and butts against the pressure member 130. As shown in FIG. 4, the handle 150 rotates relative to the housing 140, and the protrusion 151 of the handle 150 butts against the pressure member 130. Here, the cross section of the infusion tube 200 remains in the state of being flattened, that is, the inner walls of the infusion tube 200 keep close to each other, so that the fluid medicament can no longer flow in the infusion tube 200.

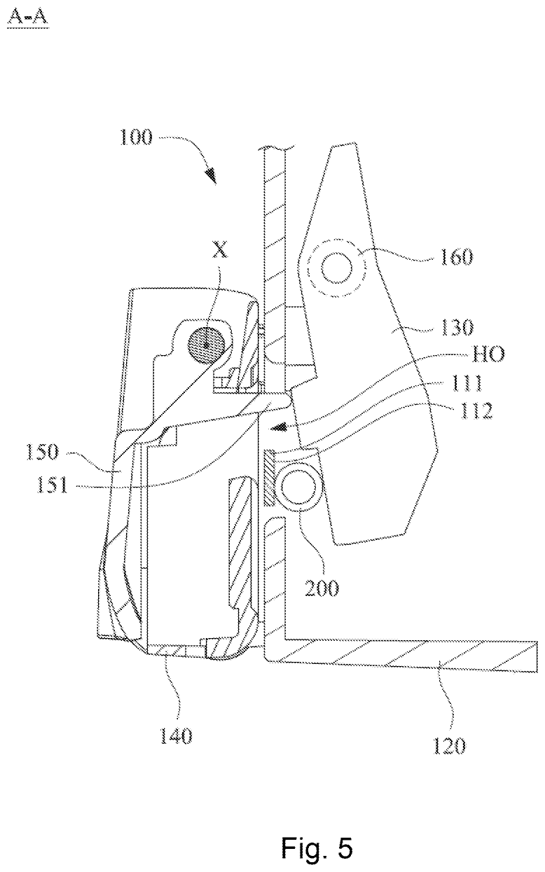

Referring to FIG. 5, it is a cross-sectional view taken along line A-A of FIG. 1, in which the protrusion 151 pushes up against the pressure member 130 to keep the pressure member 130 away from the pressure surface 112. As shown in FIG. 5, the handle 150 rotates towards the block 120 around the axis X relative to the housing 140, and the protrusion 151 of the handle 150 also pushes up against the pressure member 130 to keep the pressure member 130 away from the pressure surface 112 of the baffle wall 111. In this way, the infusion tube 200 is no longer flattened between the pressure member 130 and the pressure surface 112 thereby. Moreover, by keeping the pressure member 130 away from the pressure surface 112 of the baffle wall 111, the infusion tube 200 can return to a hollow state, so that the fluid medicament can flow in the infusion tube 200.

That is, after the infusion tube 200 passing through the clamp 110 along with the clamp 110 is inserted into and fixed in the mounting hole HM of the block 120, the infusion tube 200 is pressed against the baffle wall 111 by the pressure member 130, so that the infusion tube 200 is also maintained in the state of being flattened, so that the fluid medicament cannot flow in the infusion tube 200. Then, when the user arranges the housing 140 on the block 120 and rotates the handle 150 around the axis X relative to the housing 140, the protrusion 151 of the handle 150 rotates with the rotation of the handle 150 to push up against the pressure member 130, so that the pressure member 130 is away from the pressure surface 112 of the baffle wall 111, and the infusion tube 200 can return to the hollow state, so that the fluid medicament can flow in the infusion tube 200.

That is, the process of allowing the infusion tube 200 to flow the fluid medicament in the block 120 is conducted by the rotation of the handle 150 relative to the housing 140 after the housing 140 is disposed on the block 120, therefore, in the process of the infusion tube 200 being fixed in the block 120, the unexpected flow of the fluid medicament in the infusion tube 200 can be effectively avoided.

Further, the process of allowing the fluid medicament to flow when the infusion device 100 is started after the installation of the infusion tube 200 on the block 120 is conducted by a mechanical operation manner that the protrusion 151 of the handle 150 pushes up against the pressure member 130 so as to make the pressure member 130 away from the pressure surface 112 when the handle 150 is pulled. Therefore, the reliability of operation can be effectively improved.

Although the embodiment of FIG. 3 includes the housing 140 to which the handle 150 is pivoted, it can be understood that the handle 150 can also be detachably pivoted to the block 120, which omits the structure of the housing 140.

In addition, as shown in FIGS. 3 to 5, the infusion device 100 also includes an elastic element 160, which is covered by the pressure member 130 in the figures, so it is drawn with dotted line. The elastic element 160 elastically connects the pressure member 130 with the block 120. Before the protrusion 151 of the handle 150 is pushed up against the pressure member 130, the pressure member 130 elastically presses towards the pressure surface 112 of the baffle wall 111. In this way, after the infusion tube 200 passing through the clamp 110 along with the clamp 110 is inserted into and fixed in the mounting hole HM of the block 120, the infusion tube 200 is in the state of being pressed against the baffle wall 111 by the pressure member 130, and the infusion tube 200 thus is also maintained in the flattened state, so that the fluid medicament cannot flow in the infusion tube 200. For example, in practical applications, the elastic element 160 is a torsion spring, but the present invention is not limited thereto.

Specifically, the block 120 has an opening HO, the position of which corresponds to the pressure member 130. In other words, the pressure member 130 can be seen at least partially through the opening HO from the outside of the block 120. When the handle 150 rotates around the axis X relative to the housing 140, the protrusion 151 of the handle 150 passes through the opening HO of the block 120 and pushes up against the pressure member 130, so that the pressure member 130 is away from the pressure surface 112 of the baffle wall 111, and the infusion tube 200 can return to a hollow state, so that the fluid medicament can flow in the infusion tube 200. It should be noted that, as shown in FIG. 1, the opening HO can also be communicated with the mounting hole HM, and the position of the baffle wall 111 also corresponds to the opening HO.

Further, when the protrusion 151 of the handle 150 pushes up against the pressure member 130 so that the pressure member 130 is away from the pressure surface 112 of the baffle wall 111, the handle 150 also keeps close to the housing 140 at this time, as shown in FIG. 5. In other words, when the infusion device 100 operates, that is, when the fluid medicament flows in the infusion tube 200, the handle 150 will not excessively protrude out of the housing 140. Therefore, the risk of the user accidentally pushing against or hitting the handle 150 is effectively reduced.

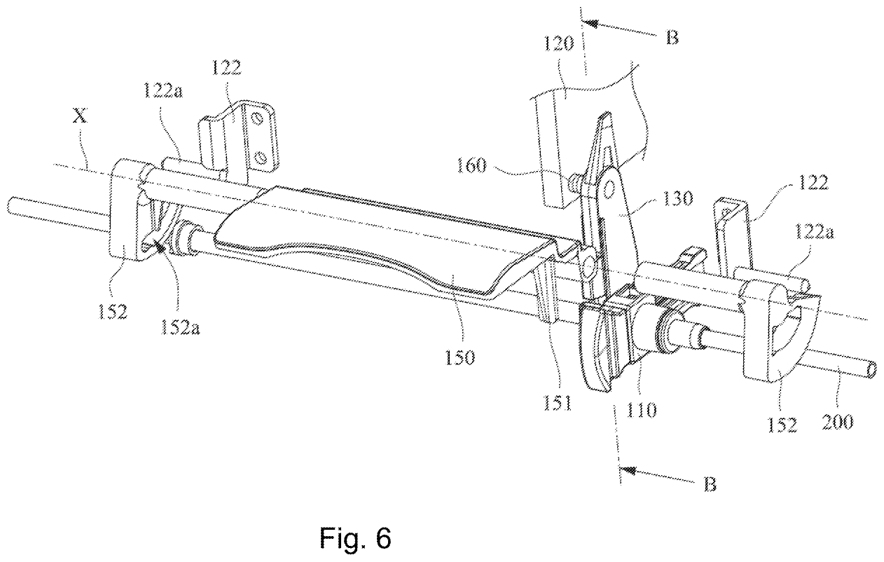

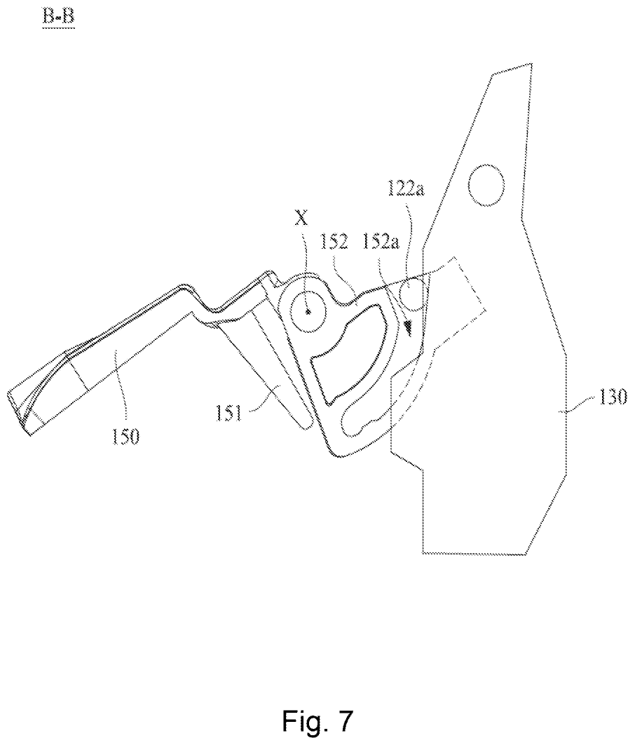

Referring to FIGS. 6-7, FIG. 6 is a partial perspective schematic view illustrating the handle 150 and the block 120 of FIG. 1, and FIG. 7 is a partial sectional view taken along line B-B of FIG. 6. In the present embodiment, as shown in FIGS. 6-7, the handle 150 also has at least a first guide portion 152, and the block 120 has at least a second guide portion 122. When the handle 150 rotates around the axis X relative to the housing 140 (see FIGS. 3-5) and before the protrusion 151 pushes up against the pressure member 130, the first guide portion 152 of the handle 150 rotates with the handle 150 and catches with the second guide portion 122 of the block 120 to fix the relative position of the housing 140 and the block 120.

In this way, before the protrusion 151 of the handle 150 pushes up against the pressure member 130 so that the pressure member 130 is away from the pressure surface 112 of the baffle wall 111, the first guide portion 152 of the handle 150 and the second guide portion 122 of the block 120 have been caught with each other to ensure that the relative position of the housing 140 and the block 120 has also been relatively fixed. Therefore, under the condition of reliability and safety, the user can press the handle 150 to rotate it, and to make the protrusion 151 of the handle 150 pass through the opening HO of the block 120 and push up against the pressure member 130 so as to keep the pressure member 130 away from the pressure surface 112 of the baffle wall 111.

In addition, for example, the included angle of the handle 150 with respect to the housing 140 before rotating relative to the housing 140 around the axis X is 0 degrees, and the included angle of the handle 150 with respect to the housing 140 after rotating relative to the housing 140 around the axis X to keep close to the housing 140 is 90 degrees, then when the included angle of the handle 150 with respect to the housing 140 is, for example, 60 degrees, the first guide portion 152 of the handle 150 and the second guide portion 122 of the block 120 are caught with each other, to fix the relative position of the housing 140 and the block 120. In practical applications, when the first guide portion 152 of the handle 150 and the second guide portion 122 of the block 120 begin to catch with each other, the included angle of the handle 150 relative to the housing 140 can also be set to other angles according to the actual situation, but the present invention is not limited to this.

In this embodiment, as shown in FIG. 6, the first guide portions 152 of the handle 150 includes arc guide grooves 152a, and the second guide portions 122 of the block 120 include guide posts 122a. When the arc guide grooves 152a rotate with the handle 150, the guide posts 122a are caught in the arc guide grooves 152a so that the first guide portions 152 of the handle 150 and the second guide portions 122 of the block 120 are caught with each other.

Moreover, the number of the first guide portions 152 of the handle 150 is a pair, and the pair of the first guide portions 152 are respectively located at opposite ends of the handle 150 along the axis X. In addition, the number of the second guide portions 122 of the block 120 is also a pair, and the second guide portions 122 are at least partially located between the first guide portions 152. In this way, since the catching positions of the first guide portion 152 and the second guide portion 122 are located at opposite ends of the handle 150 along the axis X, the handle 150 can be firmly caught in the block 120.

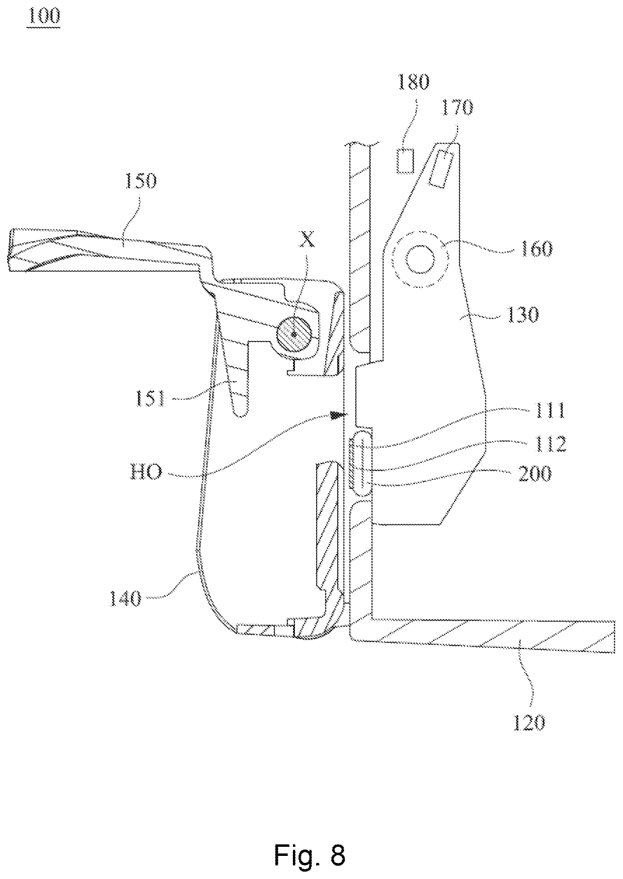

Referring to FIG. 8, it is a partial sectional view illustrating an infusion device 100 according to another embodiment of the present invention. In the present embodiment, as shown in FIG. 8, the infusion device 100 also includes a sensor 180 and a sensed object 170. The sensor 180 is located on the block 120, and the sensed object 170 is located at the other end of the pressure member 130 configured to press towards the pressure surface 112. Specifically, before the protrusion 151 of the handle 150 passes through the opening HO of the block 120 and pushes up against the pressure member 130, the positions of the sensed object 170 and the sensor 180 are staggered from each other.

However, when the protrusion 151 of the handle 150 passes through the opening HO of the block 120 and pushes up against the pressure member 130 to make the pressure member 130 away from the pressure surface 112, the sensed object 170 also rotates with the pressure member 130 to correspond to the position of the sensor 180, and the sensor 180 also senses the sensed object 170, so that the user can know that the pressure member 130 has been away from the pressure surface 112 by the signal of the sensor 180, the infusion tube 200 has returned to the hollow state, and the fluid medicament can flow in the infusion tube 200. In this way, the operational reliability of the infusion device 100 can be effectively improved.

In practical applications, the sensor 180 may be an optical sensor that senses the position of the sensed object 170 based on the optical principle. Alternatively, according to the actual situation, the sensor 180 can also be a Hall sensor, and the sensed object 170 is a magnet, but the present invention is not limited thereto.

To sum up, the technical solutions disclosed in the above embodiments of the present invention have at least the following advantages:

(1) Since the process of allowing the infusion tube to flow the fluid medicament in the block is conducted by the rotation of the handle relative to the housing after the housing is disposed on the block, the unexpected flow of the fluid medicament in the infusion tube can be effectively avoided in the process of the infusion tube being fixing in the block.

(2) The process of allowing the fluid medicament to flow when the infusion device is started after installation of the infusion tube on the block is conducted by a mechanical operation manner that the protrusion of the handle pushes up against the pressure member so as to make the pressure member away from the pressure surface when the handle is pulled. Therefore, the reliability of operation can be effectively improved.

(3) Since the first guide portion of the handle and the second guide portion of the block have been caught with each other so as to ensure that the relative position of the housing and the block has been relatively fixed before the protrusion of the handle pushes up against the pressure member to cause the pressure member away from the pressure surface of the baffle wall, the user, under the condition of reliability and safety, can press the handle to rotate it and make the protrusion of the handle pass through the opening of the block and push it up against the pressure member, so that the pressure member is away from the pressure surface of the baffle wall.

Although the present invention has been disclosed by way of illustration as above, it is not intended to limit the present invention. Any person familiar with the technical art can make various changes and refinements without departing from the spirit and scope of the present invention. Therefore, the protection scope of the present invention should be determined by the claims.

What is claimed is:

1. An infusion device comprising:
a clamp having a through hole that is configured to allow an infusion tube to pass through therethrough, and further having a baffle wall;
a block having a mounting hole and an opening, the mounting hole being configured to allow the clamp to at least partially insert and to be connected with the block;
a pressure member pivoted to the block and configured to press towards the baffle wall and close the infusion tube, wherein a position of the opening corresponds to the pressure member; and
a handle pivoted to the block and having a protrusion that is configured to pass through the opening and push up against the pressure member so as to make the pressure member away from the baffle wall when the handle rotates towards the block around an axis, so that the infusion tube is no longer closed by pressing of the pressure member.

2. The infusion device according to claim 1, characterized in that it further comprises an elastic assembly which elastically connects the pressure member and the block, wherein the pressure member elastically presses towards the pressure surface before the protrusion pushes up against the pressure member.

3. The infusion device according to claim 2, characterized in that the elastic assembly is a torsion spring.

4. The infusion device according to claim 1, characterized in that the handle is further having at least a first guide portion, the block has at least a second guide portion, and when the handle rotates relative to the housing and before the protrusion pushes up against the pressure member, the first guide portion rotates with the handle and catches with the second guide portion to fix relative position of the housing and the block.

5. The infusion device according to claim 4, characterized in that the first guide portion comprises an arc guide groove, and the second guide portion comprises a guide post that is caught in the arc guide groove when the arc guide groove rotates with the handle.

6. The infusion device according to claim 4, characterized in that the number of the first guide portions is a pair, which are respectively located at opposite ends of the handle along the axis, and the number of the second guide portions is also a pair.

7. The infusion device according to claim 1, characterized in that the handle keeps close to the housing when the protrusion pushes up against the pressure member.

8. The infusion device according to claim 1, characterized in that it further comprises a sensor that is located on the block, and a sensed object that is located at the other end of the pressure member configured to press towards the pressure surface, when the pressure member is away from the pressure surface, the sensed object corresponds to the sensor, and the sensor senses the sensed object.

9. The infusion device according to claim 8, characterized in that the sensor is a Hall sensor, and the sensed object is a magnet.
